(19) United States
(12) Patent Application Publication
(10) Pub. No.: US 2023/0105111 A1
(43) Pub. Date: Apr. 6, 2023

(54) System and Method for Teaching Minimally Invasive Interventions

(71) Applicants: Universität Heidelberg, Heidelberg (DE); DEUTSCHES KREBSFORSCHUNGSZENTRUM STIFTUNG DES ÖFFENTLICHEN RECHTS, Heidelberg (DE)

(72) Inventors: Felix NICKEL, Heidelberg (DE); Jens PETERSEN, Heidelberg (DE); Sinan ONOGUR, Heidelberg (DE); Mona SCHMIDT, Heidelberg (DE); Karl-Friedrich KOWALEWSKI, Heidelberg (DE); Matthias EISENMANN, Dossenheim (DE); Christoph THIEL, Sandhausen (DE); Sarah TRENT, Washington, DC (US); Christian WEBER, Mannheim (DE)

(21) Appl. No.: 17/908,206

(22) PCT Filed: Mar. 5, 2021
(86) PCT No.: PCT/EP2021/055674

§ 371 Date: Aug. 30, 2022

(30) Foreign Application Priority Data
Mar. 6, 2020 (EP) 20161589.5

(51) Int. Cl. G09B 23/28; G09B 5/02; G06T 7/521; G06T 7/10

(52) U.S. Cl. CPC G09B 23/285; G09B 5/02; G06T 7/521; G06T 7/10

(57) ABSTRACT

The present disclosure provides a computer implemented method for facilitating a teaching surgeon to teach or assist a learning surgeon in minimally invasive interventions using a surgical instrument including displaying endoscopic images of an intervention site in real time on a display device, said endoscopic images being captured using a camera associated with an endoscopic instrument, and tracking a movement of one or both hands of said teaching surgeon and/or a device held by said teaching surgeon, using a real-time tracking apparatus, wherein said real-time tracking apparatus comprises a tracking system and a computing device, wherein said tracking of said movement comprises recording a sequence of tracking information of one or both hands of said teaching surgeon and/or the device held by said teaching surgeon.

### FIELD OF THE INVENTION

The present invention is in the field of computer-assisted medical technology. More particularly, the present invention relates to a system and a method for teaching minimally invasive and endoscopic interventions. More broadly the invention can be used for any image-based intervention that requires working with hands and/or instruments.

### BACKGROUND OF THE INVENTION

Minimally invasive surgery, sometimes also referred to as “keyhole surgery”, is a type of surgery in which thin, rigid or flexible instruments are inserted through a small incision or possibly a natural orifice like the mouth or nostrils. At least one of the instruments, such as an endoscope or laparoscope, is equipped with a video camera which records video images from the inside of the body, which are displayed on a display apparatus. In the following disclosure, these types of the images are referred to as “endoscopic images”, irrespectively of the specific type of surgical instrument employed in the surgical intervention. The same setting can be used for interventional procedures that work with needles or catheters for and radiological image data displayed on an image apparatus.

By avoiding large incisions, minimally invasive surgery has a number of advantages over traditional, open surgery, such as less pain, lower risk of infection, shorter hospital stays, quicker recovery times and reduced blood loss

In practice, approximately 30% of all surgical interventions involve some degree of teaching a learning surgeon by an experienced, teaching surgeon. However, in minimally invasive surgery, the teaching is currently less effective than in open surgery. For example, in minimally invasive surgery the teaching surgeon cannot easily point out anatomical structures be treated or to be avoided to the learning surgeon visually, and any practical instructions for carrying out the interventions relies on verbal communication. The correct identification of the anatomy of the individual patient is believed to be the cause of at least 30% of the success of the surgery. It is reported that even up to 97% of the severe errors made during a minimally invasive gall bladder surgery were attributed to misinterpretation of the anatomy, see Way L W, Stewart L, Gantert W, Liu K, Lee C M, Whang K, et al. Causes and prevention of laparoscopic bile duct injuries: analysis of 252 cases from a human factors and cognitive psychology perspective. Ann Surg. 2003; 237(4):460-9. This demonstrates that any misunderstanding between the less experienced, learning surgeon and the more experienced, teaching surgeon in the minimally invasive intervention involves a risk to patient safety. In addition, studies show increased operative times for surgeries performed by trainee surgeons that vary between 37% to 52% additional operative time, which poses a significant cost factor for the health care sector.

Currently, there are various approaches for computer-assisted learning of surgical skills. An example for this is the app “Touch Surgery” that can be used with a smart phone or a tablet, which provides currently more than 100 different surgical simulations. Herein, in addition to the learning of the surgical steps, a possibility for an automated assessment is available. However, these apps only allow for teaching the cognitive component of surgery, but not the psychomotor skills. Also, there is no interaction between a teacher and trainee since the app relies on preprogrammed teaching software with standard situations.

On the other hand, virtual reality simulators allow for acquiring both, psychomotor and cognitive skills. Using conventional box trainers, such as the “Lübecker Toolbox”, surgical skills can be trained using real instruments and physical models. This way, the basic skills needed for minimally invasive surgery can be trained, for example sewing or tying knots, which tends to be difficult using the elongate instruments due to restricted degrees of freedom, difficult instrument coordination, restricted haptic feedback and limited working space available in minimally invasive interventions. Moreover, certain surgical steps, or even complete surgical interventions can be practiced using silicone models or animal organs. This training requires the presence of a teacher to instruct trainees and to give feedback and guidance during training. Virtual Reality (VR) simulators provide training in a virtual environment for both basic psychomotor skills to get used to the endoscopic view and instrument coordination, but also allow for training of complete virtual operative procedures. VR simulation allows for feedback but have several limitations: the realism of the VR environment is currently very limited and not suitable for more than basic training. The virtual surgeries do not adequately reflect intraoperative conditions during surgery and there is no means of using VR intraoperatively. There is some degree of skill transfer but trainees still require intraoperative guidance by experts and this does not solve the problem that there is no means of visual communication during real surgeries.

The product VIPAAR is devised for intraoperative assistance using augmented reality primarily for open surgery and uses the tablet computers, as described in Davis M C, Can D D, Pindrik J, Rocque B G, Johnston J M. Virtual Interactive Presence in Global Surgical Education: International Collaboration Through Augmented Reality. World Neurosurg. 2016; 86:103-11. A problem with this technology is that tablets are difficult to handle in the sterile environment of an operating room. Moreover, using the computer tablet display in addition to the display where the endoscopic images are displayed may lead to confusion.

### SUMMARY OF THE INVENTION

The problem underlying the invention is to provide means for facilitating the learning of minimally invasive medical and surgical interventions performed by a learning surgeon. This problem is solved by a computer implemented method for facilitating a teaching surgeon to teach or assist a learning surgeon in minimally invasive interventions according to claim 1, as well as a corresponding system according to claim 12. Preferable embodiments are defined in the dependent claims.

The present invention is based on the observation that although it is in principle possible to rely to some degree on the skills a learning surgeon has acquired in simulations outside the operation room, this does not substitute the skills obtained in real surgery. This is found to be particularly true for the preparation of tissue and for the correct intraoperative identification of the patient's anatomy, where it is seen that by the training outside the operation room alone, sufficient skills can hardly be acquired. This means that the learning surgeon still has to acquire an important share of their skills by carrying out the real intervention or at least parts thereof himself or herself, under the supervision of an experienced surgeon, who is referred to as the “teaching surgeon” in the following. The present invention aims at making this supervised surgery or intervention as efficient as possible for the learning surgeon, while keeping the additional time of the intervention as compared to the time required by an experienced surgeon low, while avoiding risk for the patient.

Accordingly, one aspect of the present invention relates to a computer implemented method for facilitating a teaching surgeon to teach or assist a learning surgeon in minimally invasive interventions using a surgical instrument. Herein, surgeon shall refer to a physician who uses operative manual and/or instrumental techniques on a person to investigate or treat a pathological condition. The term “surgical instrument” can encompass any instrument that can be used in a minimally invasive surgical intervention or endoscopic intervention, which can include but is not limited to a laparoscope/endoscope, and endoscopic scissors, graspers, needle drivers, overholts, hooks, retractors, staplers, biopsy forceps, sealing and tissue transection devices such as Ultrasonic, LigaSure, Harmonic scalpel, Sonicision or Enseal, but also needle based instruments as well as flexible and intralumenal instruments and catheters, a DJ stent, an MJ stent, a urinary catheter, guide wires, biopsy forceps, resection loops, or thulium laser. The “learning surgeon” is the surgeon currently carrying out the intervention (or a part thereof) under the supervision of the teaching surgeon. The method comprises a step of displaying endoscopic images, possibly additionally radiologic or other images, of an intervention site in real-time on a display device, said images being captured using a camera associated with an endoscopic instrument. In particular, this endoscopic instrument can be operated by the teaching surgeon. Thus, the endoscopic images can be recorded by an (endoscopic) camera. This endoscopic camera can be controlled by the teaching surgeon (with one hand). The learning surgeon operates with the other endoscopic instruments.

The method further comprises a step of tracking a movement, e.g. a two- or three-dimensional movement, of one or both hands of said teaching surgeon and/or a device held by said surgeon using a real-time tracking apparatus, wherein said real-time tracking apparatus comprises a tracking system, such as a camera or more generally a sensor, and a computing device or module. The tracking of said movement comprises the steps of
recording a sequence of tracking information of one or both hands of said teaching surgeon or a device held by said teaching surgeon, said sequence of tracking information including real-time information regarding movement of said one or both hands of the teaching surgeon or a device held by said surgeon, and
receiving, by said computing device or module, said recorded sequence of information and extracting said real-time information regarding said movement of the one or both hands or a device held by said surgeon from said recorded sequence of tracking information.

Herein, the feature that the series of tracking information “includes real-time information regarding a movement, e.g. a two- or three-dimensional movement, of the hand(s) or device” means in a broad sense that the movement is reflected in and can be extracted from the sequence of tracking information. This could for example be the case if the sequence of tracking information is a sequence of 2D images or 3D images, such as images obtained with any type of 3D camera. However, the invention is not limited to any specific type of 2D or 3D images, or to any specific way the two- or three-dimensional information is included in the tracking images, such as time-of-flight (TOF) information, stereo information, triangulation information or the like.

Recording a sequence of tracking information of one or both hands can also be performed by tracking a device held by one or both hands of the surgeon.

The movement of one or both hands is typically a three-dimensional movement. In some cases, the movement may be essentially within a plane, i.e., and thus may be considered a two-dimensional movement.

The tracking system can comprise a camera. The camera can be for example a normal 2D camera or a stereoscopic camera. The camera can be suitable for infrared imaging. Also, the camera can comprise RGB sensors. Also, the tracking system can comprise a time-of-flight camera that uses infrared light.

The surgical instrument may be for example an endoscopic surgical instrument, a cardiac catheter, or an instrument for interventional radiology.

Finally, the method comprises a step of overlaying a visual representation of the tracked movement of said one or both hands or device of the teaching surgeon over the real-time endoscopic image in an augmented reality fashion, thereby allowing the teaching surgeon to carry out gestures with one or both hands or the device which are displayed on the endoscopic image and allow for teaching or visually instructing the learning surgeon.

The overlaying of a visual representation may be triggered and stopped based on a gesture that is recognized by the system. Alternatively or additionally, the overlaying may be triggered and stopped using voice control, e.g. based on certain predefined voice commands. Gestures may also be used to trigger other actions, e.g. to start or stop the recording of the images and the overlay, or to start or stop overlaying additional images, e.g. radiologic images.

Accordingly, the method of the invention allows the teaching surgeon to make gestures with one or both of his hands and/or an additional device held in hand(s) which will be included in the image, allowing the teaching surgeon to present visual instructions or assistance to the learning surgeon. In particular, the method allows the teaching surgeon to present the visual instructions or assistance while the teaching surgeon and/or the learning surgeon are performing the surgery on the patient. In other words, there is no need to interrupt the surgery or to have an assistant. For example, the teaching surgeon could hold the endoscopic instrument with one hand, while giving visual instructions to the learning surgeon with the other hand (tracked by the real-time tracking apparatus).

For example, the teaching surgeon may point with their finger to specific anatomical structures to be prepared, to organs at risk that are to be avoided, or make a gesture indicating a line of a surgical cut to be made. Since the teaching surgeon can simply use their hand to give visual assistance, the method is very intuitive and easy to learn, and allows for the least possible distraction of the teaching surgeon, who has to be prepared to take over the endoscopic surgery instrument at any time from the learning surgeon and hence needs to focus their full attention to the surgery at all times.

Note that the conventional way to point out structures and locations in the endoscopic image would be to use a pointing device, such as a mouse, a touchpad or a trackball. However, these types of pointing devices would be difficult to handle in the sterile environment of the surgery, and the operation of such pointing device would be much more distracting for the teaching surgeon than simply using his or her hand for gestures. Moreover, since the method involves the above-mentioned information about the two- or three-dimensional movement of the hand or hands, visual representations of these two- or three-dimensional movements can likewise be displayed on the endoscopic image, allowing for example to demonstrate a sequence of complex movements to avoid a risk structure, specify where to take biopsies from suspicious tissue, tying a knot in a tight space or how to properly place a clip in order to ligate arterial and venous vessels and so on. Note that the “visual representation” of the hand(s)” can comprise the actual image of the hand extracted from the tracking information, or could comprise a computer model of a hand carrying out a two- or three-dimensional movement corresponding to the information regarding said two- or three-dimensional movement of the hand(s) as extracted from the sequence of tracking information, e.g. a sequence of tracking images. Preferably, the computer model comprises at least a representation of the five fingers and the palm of the hand. Thus, the computer model can be seen as a computer-generated life-like rendering of the hand.

In actual operating room conditions, the image that an RBG camera acquires of the hand of the teaching surgeon may be relatively dark. Thus, the method may comprise an image processing step of increasing the brightness of the RBG image of the hand before overlaying the image of the hand. There can also be a processing step of changing the color and/or transparency of the hand.

Optionally, the hand can be shown as a model in different colours and with different levels of transparency. Further, it can be visualized through a crosshair or another symbol. The hand may also be visualized through lines that remain on the screen for a short time.

Preferably, the creation of a virtual representation of the hand and does not involve a recognition of gestures, but instead refers to a virtual copy (this can be a 3D model, the real hand filtered out) of the physical hand on the screen. It can also be an abstraction that uses a model to reflects e.g. the five fingers and joints of the hand, but is not an exact image of the hand.

Preferably, the tracking of the hand is not compromised under operating room conditions because the tracking algorithm is trained to work under operating room conditions where a patient's skin or sterile drapes might be in the tracking field and these are filtered by the algorithm to not interfere with the tracking of the hand.

The virtual copy of the physical hand can be created by capturing the real hand with a suitable sensor (in particular this can be a camera sensor, e.g., but not limited to, normal RGB cameras, infrared cameras, depth cameras—e.g. stereo, time-of-flight, structured light, LIDAR). The suitability of different sensor types depends on the exact application, see example below.

The virtual representation of the hand can be generated from the sensor data. This can be, for example, an exact outline of the hand that can be displayed directly or used to isolate the hand from the captured image. Another possible representation is given by the position of a suitable set of “keypoints”, e.g. 21 joint points, which can then be used, for example, to generate a 3D model of a hand in the exact position and posture of the real hand.

The representation can be generated by suitable image processing algorithms, in particular by machine learning or deep learning. Here, a special feature is given by the fact that these algorithms have to be trained by large amounts of annotated sample data. When considering how to train such an algorithm, the following should be considered:

1) The product should be usable in real operating room operation, but for practical as well as legal reasons it may not be possible to record data in the required quantity while procedures are performed in the operating room.

2) Carrying out the method and obtaining the visual representation of the hand from sensor information can be particularly challenging because the lighting conditions vary greatly and typically only permit the use of so-called active cameras, i.e., those that themselves emit a (usually infrared) light in order to record images with it. These can include infrared and especially infrared depth cameras (ToF, Structured Light, LIDAR), but preferably do not include conventional RGB cameras.

3) Annotation of data from the above-mentioned sensor types for training purposes is generally very difficult. As a concrete example, most people are able to mark the outline or joint points of a hand in an RGB image. However, the images from the other sensors are often of poor quality and much more difficult for humans to interpret, so it is not possible to produce annotations of sufficient quality without special knowledge and a lot of practice.

Preferably, for obtaining the training data, in an accessible environment (e.g., a training center), a sensor combination is built so that an RGB sensor and a sensor (depth sensor) to be used in the operating room are superimposed, each recording the same image content (“co-registration”). Thus, one can annotate the RGB images on the acquired data (easy) and thus automatically obtain annotations for the other sensor data (difficult). In particular, this also allows using existing algorithms (deep learning models) trained only on RGB data to generate annotations for the other sensor data. These are then used to train the algorithm that will be used in the operating room. In embodiments, the visual representation of the hand can be an abstract visualization such as a point, a line or a bar.

In addition, dependent on the surgical procedure, the recognized hand model can be used to control virtual instruments on the video screen in augmented reality in order to direct the learning surgeon where to cut or to place a clip. More precisely the index and middle finger could be used as the two tranches of the scissor. This is helpful to show the correct position, angle and depth in order to avoid unintended clipping/cutting of structures at risk.

In a preferred embodiment, the tracking information comprises depth information and image information, and the method comprises: segmenting the depth information to obtain a segmentation of the hand, and extracting the actual image of the hand from the image information using the segmentation of the hand.

This has the advantage that the depth information, which may be more informative for segmentation, especially in the dark environment of an operating room, may be used to determine the segmentation, and the overlay can show the image of the hand that the users, e.g. the learning surgeon, can see on the screen.

The image information can refer e.g. to an RBG image or a grayscale image.

In a preferred embodiment, the method further comprising initial steps of: obtaining a plurality of depth information and a corresponding plurality of image information; for each of the plurality of depth information and corresponding plurality of the image information, determining a segmentation of the hand based on the image information, in order to obtain a plurality of image segmentations; and performing training of a segmentation algorithm based on the plurality of depth information and the plurality of image segmentations.

Experiments have shown that, at least when sufficient light is available, image information are easier to segment than depth information. This can apply both to manual segmentation and to segmentation by an automated algorithm.

Preferably, the above-mentioned two-step procedure can be used to enable annotation of depth images by synchronizing an RGB Sensor and a depth sensor, meaning the two sensors capture the same physical region of interest. In doing so, a person or another algorithm can be used to provide annotations for the RGB images, which then automatically become annotations for the depth images. Each recording can contain a twin/duplet comprising a RGB and a depth image. This procedure allows to train an algorithm with RGB+D images under conditions where these are available for the algorithm to then work under operating room conditions with sparse light where only depth information will be available but no RGB information.

This embodiment has the advantage that the system can be trained e.g. under bright conditions, where the segmentation of the image information is relatively easy, for an algorithm or a human operator. After training, the system can then recognize the segmentation of the hand even in absolute darkness, i.e., when only depth information is available. In a preferred embodiment, said tracking camera system is arranged or configured to be arranged close to the patient undergoing minimally invasive surgery, such that the teaching surgeon can take over the intervention from the learning surgeon at any time. The system can also be used with an additional teaching surgeon at a distance in the same room or outside of the room. This includes use as a Telementoring system at a distance.

In a preferred embodiment, said tracking camera system is attached to a supporting stand, the endoscopy tower or to an operating room light or is mounted in the room independently. This allows for meeting the sterility requirements and does not or not significantly limit the space available for the surgeons. Preferably, the tracking camera system is configured to wirelessly communicate the tracking information, such that wired cabling in the operating room is avoided.

In a preferred embodiment, the method further comprises a step of autonomously recognizing, by the computing device or module of said real-time tracking apparatus, the one or both hands or the instrument of the teaching surgeon in said series of tracking images, in particular using a machine learning algorithm. The one or both hands can potentially be located at any location in the operating room. However, the TOF camera, triangulation system stereo camera is usually placed between 25 and 75 cm above the teaching surgeon's hand(s) that is/are to be tracked. Of course different distances can be optimal for different setups and tracking devices.

In particular, the above-mentioned predetermined distance may refer to the distance above the operation situs or the distance of the hand of the surgeon to the endoscopy screen.

In a preferred embodiment, the method further comprises a step of carrying out a segmentation algorithm for extracting the hands from the recorded sequence of tracking images. In particular, RGB filters can be used for the segmentation.

In a preferred embodiment, the method further comprises a step of carrying out a segmentation algorithm for extracting the hands from the recorded sequence of tracking images. The algorithm can use, but does not require, articulated information about position and orientation of the hand(s) identified in the previous step. In particular, the algorithm is usually a machine learning algorithm, but can also be a heuristic algorithm including but not limited to color filters, texture filters, thresholding based segmentation, edge detection or region growing.

In a preferred embodiment, said tracking camera system comprises a stereo camera, a triangulation system, a time-of-flight camera or in a broader way any sensor. Each of these types of camera uses different ways to encode two- or three-dimensional information. However, each of them allows for obtaining a sequence of tracking images including real-time information regarding said two- or three-dimensional movement of the hand(s). Said camera system is preferably mounted in and/or around a sterile environment of an operating room.

In a preferred embodiment, the method comprises a step of recognizing a predetermined gesture of the one or both hands of the teaching surgeon and using the recognized predetermined gesture as a trigger for a calibrating the position and size of the visual representation of the hands overlaid to the endoscopic image. After recognition of the calibration command based on the performed gesture, the teaching surgeon can customize the following parameters based on individual preference: working field (where the hands will be positioned during the procedure), size and color of the hands presented in augmented reality on the endoscopic screen, highest and lowest point of the area in which the hands will be detected (e.g. 20 cm above the operating field), as well as format of augmented reality overlay (hands vs. procedure-specific instruments or representations of the hand, different levels of transparency of the overlaid augmented reality image). Additionally, customized settings for each surgical team can be saved on first use. Whenever needed during the procedures, those settings can be loaded without the need for a repeated calibration.

In a preferred embodiment, the visual representation of the hand or hands of the teaching surgeon is overlaid over said real-time endoscopic image as if the hand(s) were placed at a predetermined distance above the intervention site shown in the real-time endoscopic image, wherein said predetermined distance is preferably between 0 and 200 cm, preferably between 50 and 75 cm. During this process, the translated movement from the captured gesture to the endoscopic screen can, but does not need to, undergo changes in terms of magnification or speed. This means preferably the hand on the screen will be moved in the same direction as in reality.

In a preferred embodiment, the visual representation of the hand or hands of the teaching surgeon is by default shown as viewed onto the dorsum of the hand.

A further aspect of the invention relates to a system for facilitating a teaching surgeon to teach or assist a learning surgeon in minimally invasive interventions using a surgical instrument, in which endoscopic images of the intervention site are displayed in real time on a display device, said system comprising:

a real-time tracking apparatus for tracking a movement of one or both hands of said teaching surgeon and/or of a device held by said surgeon, said real-time tracking apparatus comprising
a tracking system for recording a sequence of tracking information of said hand(s) of said teaching surgeon and/or of said device held by said teaching surgeon, said tracking information including real-time information regarding a movement of said hand(s) of the teaching surgeon or said device held by said surgeon, and
a computing device or module configured for receiving said recorded sequence of information and extracting said real-time information regarding said movement of said hand(s) and/or a device held by said surgeon from said recorded sequence of information,

said system further comprising an image augmentation apparatus, wherein said image augmentation apparatus is configured for overlaying a visual representation of the tracked-movement of said hand(s) of the teaching surgeon or the device held by said surgeon with the real-time endoscopic image, thereby allowing the teaching surgeon to carry out gestures with one or both hands or a device held by said surgeon which are displayed on the endoscopic image and allow for teaching or instructing the learning surgeon.

In a preferred embodiment, said tracking system is arranged or configured to be arranged close to the patient undergoing minimally invasive surgery, such that the teaching surgeon can take over the intervention from the learning surgeon at any time.

In a preferred embodiment, said tracking system is attached to a supporting stand, an endoscopy tower, to the surgeon, to an operating room light or is mounted separately in the operating room.

In a preferred embodiment, said computing device or module of said real-time tracking apparatus is configured for autonomously recognizing the one or both hands of the teaching surgeon and/or a device held by said surgeon in said sequence of tracking information, in particular using a machine learning algorithm.

In various embodiments, the computing device or module of said real-time tracking apparatus is configured for carrying out a segmentation algorithm for extracting the hands and/or a device held by said surgeon from the recorded sequence of tracking information.

In preferred embodiments, said tracking system comprises a stereo camera, a triangulation system or a time-of-flight camera.

In preferred embodiments, said tracking system comprises a camera that is mountable in a sterile environment of an operating room.

Said computing device module is preferably configured for recognizing a predetermined gesture of the one or both hands and for using the recognized gesture as a trigger for a calibration of the position and size of the visual representation of the hands overlaid to the endoscopic image.

In various embodiments, the image augmentation apparatus is configured for overlaying the visual representation of the hand or hands of the teaching surgeon over said real-time endoscopic image as if the hand(s) were placed at a predetermined distance above the intervention site shown in the real-time endoscopic image, wherein said predetermined distance is preferably between 25 and 75 cm, preferably between 50 and 200 cm.

In preferred embodiments, the image augmentation apparatus is configured for by default showing the visual representation of the hand or hands of the teaching surgeon as viewed onto the dorsum of the hand.

### SHORT DESCRIPTION OF THE FIGURES

FIG. 1 is a schematic view of an operating room in which a system according to an embodiment of the invention is employed (left: teaching surgeon, right: learning surgeon)

FIG. 2 is a schematic view of the operating room of FIG. 1 in which the teaching surgeon is holding a dummy instrument.

FIG. 3 is an example overlay of a real-time-endoscopic image of an intervention site with a visual representation of a hand of the teaching surgeon in accordance with an embodiment of the invention.

### DESCRIPTION OF THE PREFERRED EMBODIMENTS

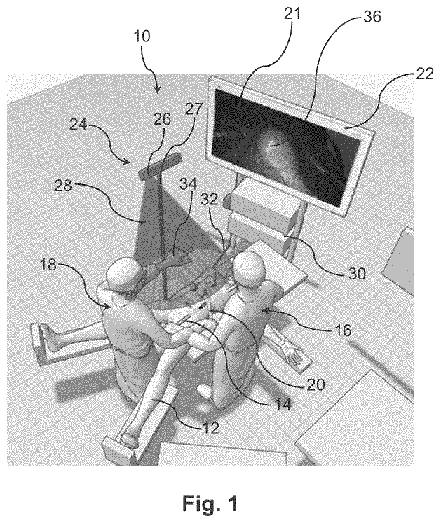

FIG. 1 is a schematic view of an operating room 10 in which a patient 12 is undergoing a laparoscopic removal of the gallbladder. The learning surgeon, i.e. the surgeon operating the instruments 14, is shown at reference sign 16 to the right in FIG. 1. The learning surgeon 16 is a less experienced surgeon, also referred to as surgeon in training, who carries out the intervention under the supervision and with the assistance of an experienced surgeon 18, also referred to as the “teaching surgeon 18” herein, standing to the left in FIG. 1. In FIG. 1, the teaching surgeon 18 operates an endoscope 20 with his right hand, to acquire endoscopic images of the intervention in real-time, which are displayed on a display device 22, such as a display screen. In other words, the endoscopic images 21 are recorded with a camera provided by the endoscope 20 operated by the teaching surgeon 18. However, in alternative embodiments, the learning surgeon 16 could operate the endoscope 20 himself or herself, or the endoscopic images 21 could be provided by a camera attached to a robotic device (not shown).

Further shown is a real-time tracking apparatus 24 generally designated by reference sign 24 in FIG. 1. The real-time tracking apparatus 24 comprises a tracking system 26, which in the embodiment shown is a 3D camera 26 operating according to the structured light principle.

In alternative embodiments, a camera that can be used that operates according to the time of flight (TOF) principle. The TOF camera 26 illuminates the scenery using a light pulse and measures, for each pixel or pixel group, the time required of the light pulse to reach the imaged object and for the light scattered by the object to return to the camera 26. Herein, the “scattered light” in particular relates to both, specularly reflected and diffusely reflected light. The required time is directly related to the distance of the respective portion of the object from the TOF camera 26. Accordingly, the images taken by the TOF camera 26, which are referred to as “tracking images” for distinguishing them from the “endoscopic images” referred to above, include real-time information regarding three-dimensional arrangement and three-dimensional movement of objects within its field of view. The region covered by the light pulses, or in other words, the field of view of the TOF camera 26, is schematically shown by the light cone 28 in FIG. 1.

As is further shown in FIG. 1, the teaching surgeon 18 holds his left hand 34 in the field of view (light cone 28) of the TOF camera 26, such that the tracking images taken include information regarding three-dimensional movement of the teaching surgeon's left hand 34. In the embodiment shown in FIG. 1, the camera 26 is attached to a supporting stand 27.

The real-time tracking apparatus 24 further comprises a computing device 30 connected with the TOF camera 26 by a cable 32, for conveying a recorded sequence of tracking images. Herein, the “sequence of tracking images” can have a frame rate of for example 30 images per second, such that the sequence of images can be regarded as a video stream. In the present embodiment, computing device 30 comprises a software module for extracting the real-time information regarding the movement of the teaching surgeon's 18 left hand 34 from the sequence of tracking images provided by the TOF camera 26. In the embodiment shown, the computing device 30 comprises an ordinary microprocessor for carrying out the computations under suitable program control. While in the present embodiment, extraction of the hand 34 from the sequence of tracking images is carried out by a software module provided on an essentially general purpose computer 30, the invention is not limited to this. Instead, the extraction function can be carried out by any combination of hardware and software.

Moreover, the computing device 30 further comprises an augmentation module, i.e. a software module which is configured for overlaying a visual representation 36 of the extracted three-dimensional movement of the teaching surgeon's 18 hand 34 over the real-time endoscopic image 21 displayed on display 22 in an augmented reality fashion. The essentially general purpose computer 30 equipped with the augmentation module hence forms a representation of the “image augmentation apparatus” referred to above. The image augmentation apparatus can be embodied in any suitable combination of hardware and/or software.

Next, the function of the system shown in FIG. 1 is described in more detail. Using the system of FIG. 1, the learning surgeon 16 can carry out the minimally invasive intervention completely or partly by himself, by operating the endoscopic surgical instrument 14, hence representing the “learning surgeon” for at least part of the intervention. Both, the teaching surgeon 18 and the learning surgeon 16 monitor the intervention by means of the endoscopic images 21 acquired with the endoscope 20 and shown on the display device 22. The experienced surgeon 18 can give not only verbal instructions and assistance to the learning surgeon 16, but can also provide visual assistance directly in the endoscopic image, by means of their left hand 34 which is projected into the endoscopic image. More precisely, a visual representation 36 of the three-dimensional movement of the teaching surgeon's 18 hand 34 is overlaid over the endoscopic image 21, such that the teaching surgeon 18 can move their hand and this movement is displayed in the augmented endoscopic image 21 as if the teaching surgeon would put their hand inside the body, close to the intervention site. Since the recording of the tracking images, extraction of the information regarding three-dimensional movement of the hand 34 and the augmentation is carried out in real-time, the augmented visual representation 36 of the hand 34 follows the real movement of the hand 34 practically simultaneously, such that the teaching surgeon 18 receives immediate visual feedback and can easily guide the visual representation 36 of their hand 34 through the endoscopic image 21.

This way, the teaching surgeon 18 can provide very useful visual assistance to the learning surgeon 18 that cannot be easily communicated verbally. For example, the teaching surgeon 18 can point out certain organs or structures to be excised or to be spared, the placement and orientation of an incision or cut to be made, the entrance and exit points of instruments or needles in the tissue, or even mimic maneuvers that could be difficult to carry out in view of the cumbrous elongate instruments and limited space. Both, the learning and the teaching surgeon 16, 18 can fully concentrate on what is seen on the display 22, and in principle, no further equipment is needed that would distract the teaching surgeon 18 and that would be difficult to handle in the sterile environment.

The operation of the system is intuitive and easy to use for the teaching surgeon 18. Moreover, the visual assistance provided by the system is much more valuable than e.g. pointing to image on the display screen 22 with his or her hand, which is very imprecise due to the distance to the display screen 22, or pointing to the image using a pointing device such as a mouse, a touchpad or a trackball, as this would distract the teaching surgeon's 18 attention, would be difficult to handle in the sterile environment, and would not allow for providing the same degree of intelligible information that can be taken from a visual representation of hand gestures based on information regarding the two- and three-dimensional movement of the hand 34. That is to say, although the image 21 displayed on the display 22 is generally two-dimensional, the three-dimensional movement of the hand 34 is still provided in a manner that can be easily understood by the learning surgeon 16. For example, when teaching surgeon 18 moves their hand 34 forward, this corresponds to a movement into the image plane of the endoscopic image 21, and this can be easily recognised from the video stream of augmented endoscopic images, even though the images per se are two-dimensional only in conventional endoscopic displays. Three-dimensional displays with three-dimensional gesture guidance by means of the described system will also be possible.

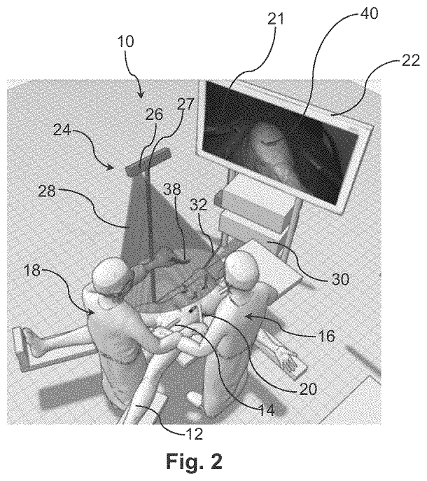

In addition, or as an alternative to the teaching surgeon's hands, a dummy instrument (e.g. a 3-D printed oversized curved needle) can be used to guide the learning surgeon (see FIG. 2, with the dummy instrument 38, and a visual representation 40 of the dummy instrument).

Due to the improved assistance that can be provided by the teaching surgeon 16, the learning surgeon 18 can be put in a position to carry out more difficult tasks himself or herself, without an increased risk for the patient 12 or one excessive prolongation of the intervention. Moreover, since the teaching surgeon 18 is not distracted by providing the visual input, he/she can devote his/her full attention to the intervention, and recognize errors before they occur, give further input, or take over control of the surgical instrument 14 from the learning surgeon 16.

The system may be initially calibrated. This can be adjustable in such a way that the pointing surgeon with his pointing (to be tracked) hand can determine a center point, a plane and the extremes of this plane within the scope of the calibration. There should be standard presets with tracking field sizes/angles/levels that have proven themselves in evaluations. It should also be possible during surgery to change the position in the room and at the operating table and then recalibrate.

The system and method of the invention thereby allow for increasing the learning effect for the learning surgeon 16, while keeping the intervention times shorter than without the visual instruction provided by the invention, and without increasing the risk for the patient 12.

Dummy instruments, e.g. a large needle, can be used as pointing devices. These devices can be used to train a segmentation algorithm, such that they can be recognized in images acquired by the tracking system.

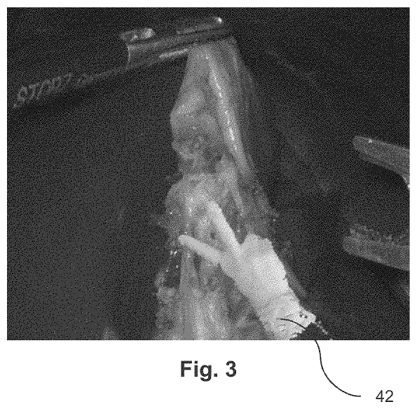

FIG. 3 shows an overlay of the endoscopic image and a visual representation 42 of the hand of the teaching surgeon, wherein the hand of the teaching surgeon is making a scissor-like movement with middle finger and pointing finger. FIG. 3 shows the freely dissected hilus of the gallbladder with exposed cystic artery. The gallbladder is lifted and clamped with the left instrument. The right instrument has the clipper ready, with which a metal clip is placed on the cystic artery in such a way that it is alloyed. The virtual hand of the teaching surgeon shows where the clip should be placed on the artery. In the background is the liver, to which the gallbladder is still attached and will be detached (dissected) during the operation.

Instead of the hand, also a tip of the instrument can be shown. For example, the two blades of scissors can be shown and a movement of middle finger and index finger can simulate a movement of the blades of the scissors (see FIG. 3). The system can be configured to interpret this as a command that the teaching surgeon now wants to use scissors as “virtual assistance”. A pair of scissors would then be displayed instead of the hand. Internally, the system then transfers the movements of the hand to the displayed scissors. For example, the hand model can be transferred in such a way that the teaching surgeon can control the scissors with his or her index and middle finger. In particular, an algorithm can be provided to segment the fingers from the acquired images. Preferably, the scissors themselves are not automatically recognized, but would have to be selected via a menu.

1. A computer implemented method for facilitating a teaching surgeon to teach or assist a learning surgeon in minimally invasive interventions using a surgical instrument (14), said method comprising:
displaying endoscopic images of an intervention site in real time on a display device, said endoscopic images being captured using a camera associated with an endoscopic instrument,
tracking a movement of one or both hands of said teaching surgeon and/or a device held by said teaching surgeon, using a real-time tracking apparatus, wherein said real-time tracking apparatus comprises a tracking system and a computing device, wherein said tracking of said movement comprises:
recording a sequence of tracking information of one or both hands of said teaching surgeon and/or the device held by said teaching surgeon, said sequence of tracking information including real-time information regarding a movement of said one or both hands of the teaching surgeon and/or said device held by said teaching surgeon, and
receiving, by said computing device or module, said recorded sequence of tracking information and extracting said real-time information regarding said movement of the one or both hands or the device held by said teaching surgeon from said recorded sequence of tracking information, and
overlaying a visual representation of the tracked movement of said one or both hands or said device held by said teaching surgeon over the real-time endoscopic image, thereby allowing the teaching surgeon to carry out gestures with one or both hands which are displayed on the endoscopic image and allow for teaching or instructing the learning surgeon.

2. The method of claim 1, wherein the visual representation of the hand is an actual image of the hand extracted from the tracking information and/or a life-like rendering of the hand determined based on the tracking information.

3. The method of 2, wherein the tracking information comprises depth information and image information, and wherein the method comprises:
segmenting the depth information to obtain a segmentation of the hand, and
extracting the actual image of the hand from the image information using the segmentation of the hand.

4. The method of claim 1, further comprising:
obtaining a plurality of depth information and a corresponding plurality of image information,
for each of the plurality of depth information and corresponding plurality of the image information, determining a segmentation of the hand based on the image information, in order to obtain a plurality of image segmentations, and
performing training of a segmentation algorithm based on the plurality of depth information and the plurality of image segmentations.

5. The method of claim 1, wherein said tracking system is arranged or configured to be arranged close to the patient undergoing minimally invasive surgery, such that the teaching surgeon can take over the intervention from the learning surgeon at any time.

6. The method of claim 1, wherein said tracking system is attached to a supporting stand, an endoscopy tower, to the teaching surgeon, to an operating room light, or is mounted independently in the operating room.

7. The method of claim 1, further comprising autonomously recognizing, by the computing device or module of said real-time tracking apparatus, the one or both hands of the teaching surgeon or the device held by said teaching surgeon in said sequence of tracking information, in particular using a machine learning algorithm.

8. The method claim 1, wherein said tracking system comprises a stereo camera, a structured light camera system, a triangulation system, or a time-of-flight camera.

9. The method of claim 1, wherein said system is mounted in a sterile environment of an operating room and/or in an environment representing an operating room for training and/or scientific purposes.

10. The method of claim 1, further comprising recognizing a predetermined gesture of the one or both hands of the teaching surgeon and using the recognized predetermined gesture as a trigger for calibrating a position and size of the visual representation of the hand(s) overlaid to the endoscopic image.

11. The method of claim 1, wherein the visual representation of the hand or hands of the teaching surgeon is overlaid over said real-time endoscopic image as if the hand(s) were placed at a predetermined distance above the intervention site shown in the real-time endoscopic image and/or wherein the visual representation of the hand(s) of the teaching surgeon is by default shown as viewed onto the dorsum of the hand, wherein preferably when the hand is turned the appearing side is shown in the visual representation.

12. A system for facilitating a teaching surgeon to teach or assist a learning surgeon in minimally invasive interventions using a surgical instrument, in which endoscopic images of an intervention site are displayed in real time on a display device, said system comprising:
a real-time tracking apparatus for tracking a movement of one or both hands of said teaching surgeon and/or a device held by said teaching surgeon, said real-time tracking apparatus comprising:
a tracking system for recording a sequence of tracking information of one or both hands and/or said device held by said teaching surgeon, said sequence of tracking information including real-time information regarding a movement of said one or both hands and/or said device, and
a computing device or module configured for receiving said recorded sequence of information and extracting said real-time information regarding said movement of said one or both hands and/or said device held by said teaching surgeon from said recorded sequence of information,
said system further comprising an image augmentation apparatus, wherein said image augmentation apparatus is configured for overlaying a visual representation of the tracked movement of said one or both hands and/or said device held by said teaching surgeon over the real-time endoscopic image, thereby allowing the teaching surgeon to carry out gestures with one or both hands which are displayed on the endoscopic image and allow for teaching or instructing the learning surgeon.

13. The system of claim 12, wherein
said tracking system is attached to a supporting stand, an endoscopy tower, to the teaching surgeon or to an operating room light, or is mounted independently in an operating room,
said tracking system comprises a stereo camera, a structured light camera system, a triangulation system, or a time-of-flight camera, and/or
said tracking system comprises a camera that is mountable in a sterile environment of the operating room and/or in an environment representing an operating room for training and/or scientific purposes.

14. The system of claim 12, wherein
said computing device or module of said real-time tracking apparatus is configured for autonomously recognizing the one or both hands of the teaching surgeon in said sequence of tracking information, in particular using a machine learning algorithm, and/or
the computing device or module of said real-time tracking apparatus is configured for carrying out a segmentation algorithm for extracting the hand(s) from the recorded sequence of tracking information.

15. The system of claim 12, wherein
the image augmentation apparatus is configured for overlaying the visual representation of the hand or hands of the teaching surgeon over said real-time endoscopic image as if the hand(s) were placed at a predetermined distance above the intervention site shown in the real-time endoscopic image, and/or
the image augmentation apparatus is configured for by default showing the visual representation of the hand or hands of the teaching surgeon as viewed onto the dorsum of the hand.
